(19) United States
(12) Patent Application Publication
(10) Pub. No.: US 2013/0167843 A1
(43) Pub. Date: Jul. 4, 2013

(54) PIEZOELECTRIC BLOWER PILOTED VALVE

(71) Applicants: Gardner Kimm, Carlsbad, CA (US); Peter Doyle, Vista, CA (US)

(72) Inventors: Gardner Kimm, Carlsbad, CA (US); Peter Doyle, Vista, CA (US)

(73) Assignee: Nellcor Puritan Bennett LLC, Boulder, CO (US)

(21) Appl. No.: 13/341,956

(22) Filed: Dec. 31, 2011

(51) Int. Cl. A61M 16/20; F16K 31/02

(52) U.S. Cl. 128/205.24; 137/487.5

(57) ABSTRACT

This disclosure describes systems and methods for piloting a pneumatic valve using one or more piezoelectric blowers. According to embodiments, the one or more piezoelectric blowers may be coupled to the pneumatic valve to form a small, light-weight pneumatic valve that may be placed proximal to a ventilated patient, e.g., at the patient wye or the patient interface. Due to the close coupling of the one or more piezoelectric blowers, the pneumatic valve has a substantially shorter response time than traditional pneumatically piloted valves. Moreover, when piezoelectric blowers are coupled to the pneumatic valve in parallel, response time may be further decreased. Additionally or alternatively, when piezoelectric blowers are coupled to the pneumatic valve in series, pilot pressure may be increased as a function of the number of piezoelectric blowers in the series.

### INTRODUCTION

A ventilator is a device that mechanically helps patients breathe by replacing some or all of the muscular effort required to inflate and deflate the lungs. Ventilators generally comprise a number of components for delivering ventilation to patients. These components include, at least, a pressure-generating source (e.g., compressor), patient tubing and patient interfaces for providing breathing gases to patients, valves and other regulatory devices for regulating the pressure and/or volume of the breathing gases, etc. Traditional valves include pneumatically piloted valves. However, traditional pneumatically piloted valves are bulky because they require a compressor or other source of pressurized gas. Generally, the pressurized gas source is placed some distance from the patient and, as such, pneumatically piloted valves are either placed near the pressurized gas source (i.e., some distance from the patient) or placed near the patient (i.e., some distance from the gas source). When pneumatically piloted valves are placed some distance from the gas source, tubing or other connectors are required that increase resistance and a corresponding response time of the pneumatically piloted valve. When pneumatically piloted valves are placed some distance from the patient, additional tubing can cause patient discomfort and reduced patient compliance, especially in the case of non-invasive patient interfaces that require additional tubing to a distal pneumatic valve.

As such, pneumatically piloted valves have been largely replaced by electrical-mechanical valves in ventilators. However, electrical-mechanical valves are also bulky and are ill-suited for proximal placement. Indeed, clinicians and patients may greatly benefit from pneumatically piloted valves coupled to one or more small, light-weight piezoelectric blowers.

### Piezoelectric Blower Piloted Valve

This disclosure describes systems and methods for piloting a pneumatic valve using one or more piezoelectric blowers. According to embodiments, the one or more piezoelectric blowers may be coupled to the pneumatic valve to form a small, light-weight pneumatic valve that may be placed proximal to a ventilated patient, e.g., at the patient wye or the patient interface. Due to the coupling of the one or more piezoelectric blowers, the pneumatic valve has a substantially shorter response time than traditional pneumatically piloted valves. Moreover, when piezoelectric blowers are coupled to the pneumatic valve in parallel, response time may be further decreased. Additionally or alternatively, when piezoelectric blowers are coupled to the pneumatic valve in series, pilot pressure may be increased as a function of the number of piezoelectric blowers in the series.

According to further embodiments, a piezoelectric blower piloted valve may be incorporated into a ventilatory system. For example, a piezoelectric blower piloted valve may be used as an exhalation valve, safety valve or other suitable valve in a ventilatory system. Moreover, due to the small size and light weight of a piezoelectric blower piloted valve, the valve may be placed proximal to the patient, e.g., at a patient wye and/or a patient interface. For example, a piezoelectric blower piloted valve may be provided in a non-invasive patient interface for regulating exhaled gases and thereby reducing re-breathing.

According to embodiments, a pneumatic valve is provided. The pneumatic valve comprises a valve housing surrounding an internal pneumatic valve chamber, the internal pneumatic valve chamber divided by a diaphragm into a plurality of chambers. The plurality of chambers comprise an inlet chamber having a valve inlet for receiving gases, the inlet chamber having an inlet pressure exerting an inlet force on the diaphragm; and a pilot pressure chamber coupled to a piezoelectric outlet port for receiving gases, the pilot pressure chamber having a pilot pressure exerting a pilot force on the diaphragm. A piezoelectric blower is coupled to the pneumatic valve, the piezoelectric blower having a piezoelectric inlet port for receiving gases and the piezoelectric outlet port for delivering pressurized gases to the pilot pressure chamber. The pneumatic valve further comprises a valve seat disposed within the inlet chamber and the diaphragm flexibly displaced based on the pilot force and the inlet force.

According to further embodiments, a method for delivering ventilation to a patient is provided. The method comprises delivering inspiratory gases to a patient during an inspiratory phase and regulating a pneumatic exhalation valve during the inspiratory phase. The step of regulating the pneumatic exhalation valve comprises receiving gases into an inlet chamber of the pneumatic exhalation valve, wherein an inlet pressure exerts an inlet force on the diaphragm of the pneumatic exhalation valve based on an area of a valve seat. The step of regulating the pneumatic exhalation valve further comprises controlling a piezoelectric blower to deliver pressurized gases to a pilot pressure chamber, wherein a pilot pressure exerts a pilot force on the diaphragm of the pneumatic exhalation valve based on an area of the diaphragm. The method further comprises substantially closing the pneumatic exhalation valve when the pilot force is greater than the inlet force.

According to further embodiments, a pneumatic valve means is provided. The pneumatic valve means comprises a valve housing means surrounding an internal pneumatic valve chamber, the internal pneumatic valve chamber divided by a diaphragm means into a plurality of chambers. The plurality of chambers comprises an inlet chamber having a valve inlet for receiving gases, the inlet chamber having an inlet pressure exerting an inlet force on the diaphragm means, and a pilot pressure chamber coupled to a piezoelectric outlet port for receiving gases, the pilot pressure chamber having a pilot pressure exerting a pilot force on the diaphragm means. The pneumatic valve means further comprises a piezoelectric blower means coupled to the pneumatic valve means, the piezoelectric blower means having a piezoelectric inlet port for receiving gases and the piezoelectric outlet port for delivering pressurized gases to the pilot pressure chamber. The pneumatic valve means further comprises a valve seat means disposed within the inlet pressure chamber and the diaphragm means flexibly displaced based on the pilot force and the inlet force.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the claims.

### BRIEF DESCRIPTION OF THE DRAWINGS

The following drawing figures, which form a part of this application, are illustrative of described technology and are not meant to limit the scope of the claims in any manner, which scope shall be based on the claims appended hereto.

FIG. 1 is a diagram illustrating an embodiment of an exemplary ventilator connected to a human patient.

FIG. 2 is a diagram illustrating a piezoelectric blower coupled to a pneumatic valve in a closed position.

FIG. 3 is a diagram illustrating a piezoelectric blower coupled to a pneumatic valve in an open position.

FIG. 4 is a diagram illustrating a plurality of piezoelectric blowers coupled in parallel to a pneumatic valve in an open position.

FIG. 5 is a diagram illustrating a plurality of piezoelectric blowers coupled in series to a pneumatic valve in a closed position.

FIG. 6 is a flow chart illustrating an embodiment of a method for delivering ventilation to a patient using an exhalation valve piloted with a piezoelectric blower.

### DETAILED DESCRIPTION

Although the techniques introduced above and discussed in detail below may be implemented for a variety of medical devices, the present disclosure will discuss the implementation of these techniques for use in a mechanical ventilator system. The reader will understand that the technology described in the context of a ventilator system could be adapted for use with other therapeutic equipment having pneumatically-piloted valves.

This disclosure describes systems and methods for piloting a pneumatic valve using one or more piezoelectric blowers. According to embodiments, the one or more piezoelectric blowers may be coupled to the pneumatic valve to form a small, light-weight pneumatic valve that may be placed proximal to a ventilated patient, e.g., at the patient wye or the patient interface. Due to the close coupling of the one or more piezoelectric blowers, the pneumatic valve has a substantially shorter response time than traditional pneumatically piloted valves. Moreover, when piezoelectric blowers are coupled to the pneumatic valve in parallel, response time may be further decreased. Additionally or alternatively, when piezoelectric blowers are coupled to the pneumatic valve in series, pilot pressure may be increased as a function of the number of piezoelectric blowers in the series.

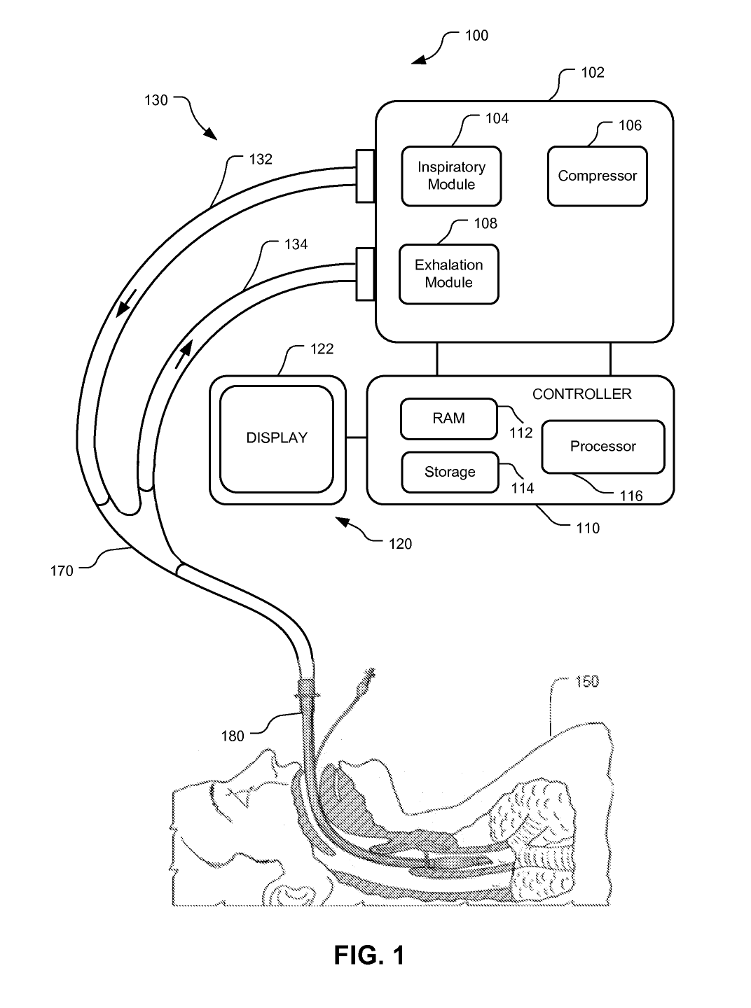

FIG. 1 is a diagram illustrating an embodiment of an exemplary ventilator 100 connected to a human patient 150.

Ventilator 100 includes a pneumatic system 102 (also referred to as a pressure generating system 102) for circulating breathing gases to and from patient 150 via the ventilation tubing system 130, which couples the patient to the pneumatic system via an invasive (e.g., endotracheal tube, as shown) or a non-invasive (e.g., nasal mask) patient interface.

Ventilation tubing system 130 may be a two-limb (shown) or a one-limb circuit for carrying gases to and from the patient 150. In a two-limb embodiment, a fitting, typically referred to as a “wye-fitting” 170, may be provided to couple a patient interface 180 (as shown, an endotracheal tube) to an inspiratory limb 132 and an expiratory limb 134 of the ventilation tubing system 130.

Pneumatic system 102 may be configured in a variety of ways. In the present example, system 102 includes an exhalation module 108 coupled with the expiratory limb 134 and an inhalation module 104 coupled with the inspiratory limb 132. Compressor 106 or other source(s) of pressurized gases (e.g., air, oxygen, and/or helium) is coupled with inhalation module 104 to provide a gas source for ventilatory support via inspiratory limb 132.

The pneumatic system 102 may include a variety of other components, including mixing modules, valves, sensors, tubing, accumulators, filters, etc. Controller 110 is operatively coupled with pneumatic system 102, signal measurement and acquisition systems, and an operator interface 120 that may enable an operator to interact with the ventilator 100 (e.g., change ventilatory settings, select operational modes, view monitored parameters, etc.). Controller 110 may include memory 112, one or more processors 116, storage 114, and/or other components of the type commonly found in command and control computing devices. In the depicted example, operator interface 120 includes a display 122 that may be touch-sensitive and/or voice-activated, enabling the display 122 to serve both as an input and output device.

According to embodiments, the pneumatic system 102 may include one or more pneumatic valves (not shown). For example, the pneumatic system 102 may control the one or more pneumatic valves to regulate the pressure and/or flow of gases. According to embodiments, a pneumatic exhalation valve may be associated with the exhalation module 108 in order to release exhaust gases from the patient 150 or to release excess gases from the pneumatic system 102. For example, the pneumatic exhalation valve may be controlled according to a trajectory to target a positive end expiratory pressure (PEEP) at the end of exhalation. According to additional embodiments, the pneumatic exhalation valve may be activated to regulate the pressure or flow of inspiratory gases to the patient 150. For example, the pneumatic exhalation valve may be controlled to release excess pressure whenever pressure exceeds a target inspiratory pressure. According to further embodiments, the one or more pneumatic valves may further include a safety valve for releasing excess pressure from the pneumatic system 102, e.g., in the event of a patient cough. According to other embodiments, the one or more pneumatic valves may include any other suitable valve for regulating gases in the pneumatic system 102, e.g., pneumatic valves associated with gas delivery, gas diversion (e.g., for purposes of evaluation), or gas release.

According to further embodiments, the one or more pneumatic valves may be closely coupled to one or more piezoelectric blowers for piloting pressure within the one or more pneumatic valves. As used herein, the phrase “closely coupled” means affixed substantially directly to a pilot pressure chamber of a pneumatic valve or affixed substantially directly to another piezoelectric blower in series. As used herein, “piloting pressure” means regulating gas pressure within a pilot pressure chamber to open or close a pneumatic valve. According to embodiments, a pneumatic valve piloted by one or more piezoelectric blowers (i.e., a piezoelectric blower piloted valve) is a small, light-weight valve that may be located proximal to the patient. That is, a piezoelectric blower piloted valve may be placed at the patient wye or within or near a patient interface.

According to some embodiments, a piezoelectric blower piloted valve may be coupled to or incorporated into a non-invasive (NIV) patient interface. As a result of the cumbersome tubing and slow response time of traditional pneumatic valves, NIV interfaces have traditionally incorporated a passive exhalation vent utilizing a fixed orifice open to ambient. However, passive exhalation vents do not consistently prevent re-breathing of exhaled air. Accordingly, a piezoelectric blower piloted valve may be used to regulate exhaled gases and to prevent re-breathing in a NIV interface.

The memory 112 includes non-transitory, computer-readable storage media that stores software that is executed by the one or more processors 116 and which controls the operation of the ventilator 100. In an embodiment, the memory 112 includes one or more solid-state storage devices such as flash memory chips. In an alternative embodiment, the memory 112 may be mass storage connected to the one or more processors 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the one or more processors 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

Communication between components of the ventilatory system or between the ventilatory system and other therapeutic equipment and/or remote monitoring systems may be conducted over a distributed network, as described further herein, via wired or wireless means. Further, the present methods may be configured as a presentation layer built over the TCP/IP protocol. TCP/IP stands for “Transmission Control Protocol/Internet Protocol” and provides a basic communication language for many local networks (such as intra- or extranets) and is the primary communication language for the Internet. Specifically, TCP/IP is a bi-layer protocol that allows for the transmission of data over a network. The higher layer, or TCP layer, divides a message into smaller packets, which are reassembled by a receiving TCP layer into the original message. The lower layer, or IP layer, handles addressing and routing of packets so that they are properly received at a destination.

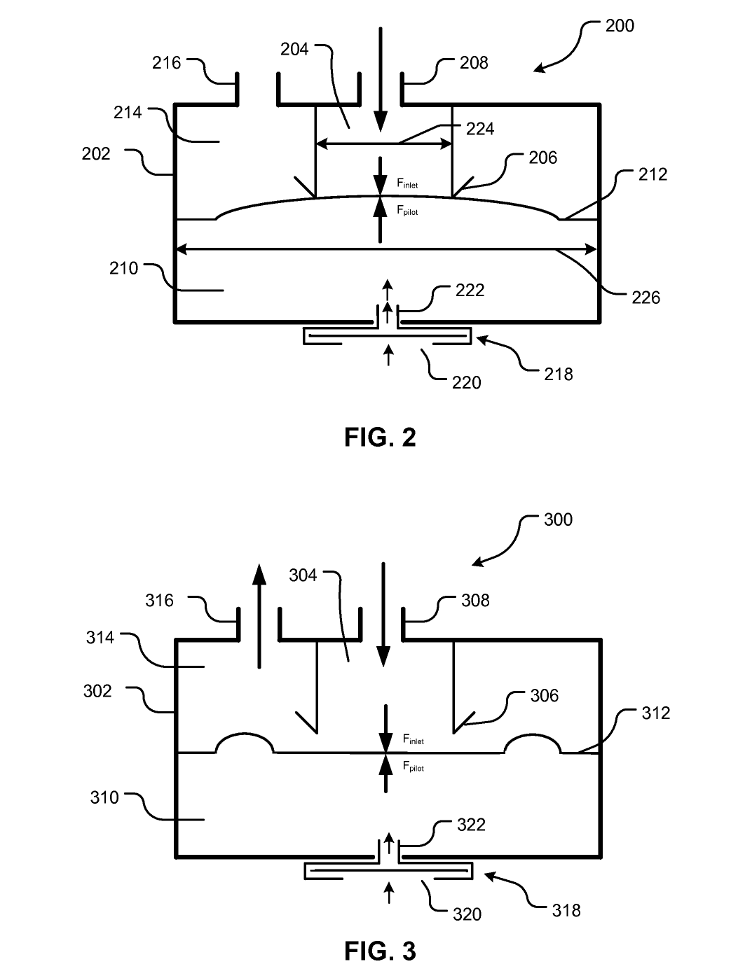

FIG. 2 is a diagram illustrating a piezoelectric blower coupled to a pneumatic valve in a closed position.

As illustrated, pneumatic valve 200 comprises a valve housing 202 that surrounds an internal pneumatic valve chamber. The internal pneumatic valve chamber is further divided into a number of additional chambers. For example, the internal pneumatic valve chamber comprises an inlet chamber 204. The volume of the inlet chamber 204 is defined by a valve seat 206 and gas enters the inlet chamber 204 through valve inlet 208. According to embodiments, the gas may be exhaled gas from a patient, excess inspiratory gas delivered by a ventilator (e.g., pneumatic system 102), or any other appropriate gas source.

According to embodiments, the internal pneumatic valve chamber further comprises a pilot pressure chamber 210. The pilot pressure chamber 210 is separated from other chambers by a diaphragm 212 that is affixed within the pneumatic valve 200. Diaphragm 212 may be made of any suitable material such that diaphragm 212 comprises both rigid and flexible characteristics, e.g., silicone.

According to embodiments, the internal pneumatic valve chamber further comprises an outlet chamber 214. When the pneumatic valve 200 is in an open position, gas entering the inlet chamber 204 is allowed to enter the outlet chamber 214. When the pneumatic valve 200 is in a closed position, gas entering the inlet chamber 204 is not allowed to enter the outlet chamber 214. Gas exits the outlet chamber 214 through valve outlet 216. Gas exiting the pneumatic valve 200 through the valve outlet 216 may be released to the atmosphere, to the expiratory limb of the patient tubing, to the expiratory module of pneumatic system 102, or to another chamber suitable for releasing gases from the pneumatic valve 200.

According to further embodiments, pneumatic valve 200 includes a piezoelectric blower 218, A piezoelectric blower is a small, electrically-powered device that generates pressurized gases. For example, an exemplary piezoelectric blower may be about 20 millimeters (mm) wide by 20 mm long by 1.85 mm thick and may weigh only about 1 gram. According to embodiments, a source of electric current excites a piezoelectric crystal to vibrate within a piezoelectric blower. Based on a speed of vibration of the piezoelectric crystal, gas is forced through an outlet port of the piezoelectric blower at an increased pressure of up to about 30 cmH₂O. According to embodiments, increasing the electric current to the piezoelectric blower causes the piezoelectric crystal to vibrate faster, generating a higher gas pressure; whereas decreasing the electric current to the piezoelectric blower causes the piezoelectric crystal to vibrate more slowly, generating a lower gas pressure. An exemplary piezoelectric blower is the MZB 1001 piezoelectric blower manufactured by Murata Manufacturing Co., Ltd., of Japan.

Traditionally, pneumatic valves are connected to a source of pressurized gas, e.g., a compressor, via tubing or other connectors. Tubing and connectors create additional pneumatic resistance and capacitance, which in turn decreases the response time of traditional pneumatic valves. As a result, many ventilators use a type of electrical-mechanical valve apparatus. An electrical-mechanical valve comprises a type of actuator, e.g., a voice coil, connected to a source of electric current. Increasing current to the voice coil causes the voice coil to engage a diaphragm, thereby closing the valve. However, an electrical-mechanical valve is relatively large, e.g., 1 inch wide by 1 inch long by 1 inch thick. Moreover, the voice coil apparatus may be relatively heavy in comparison to a piezoelectric blower piloted valve.

According to embodiments, piezoelectric blower 218 is coupled to pneumatic valve 200 via any suitable means, e.g., via tubing, a connector, an interface, etc. According to some embodiments, piezoelectric blower 218 is closely coupled to pneumatic valve 200. That is, it is not necessary to connect the piezoelectric blower 218 to the pneumatic valve 200 via tubing or other connector. Rather, the piezoelectric blower 218 may be affixed substantially directly to pneumatic valve 200. According to embodiments, “affixed substantially directly” comprises any suitable gas-impermeable barrier or interface for closely coupling the piezoelectric blower to pneumatic valve 200.

According to embodiments, gas enters piezoelectric blower 218 through a piezoelectric inlet port 220. For example, the piezoelectric inlet port 220 may be open to the atmosphere, ventilatory tubing, or any other suitable source of gas. Pressurized gases exit the piezoelectric blower 218 through a piezoelectric outlet port 222 that leads into the pilot pressure chamber 210.

In general, an inlet pressure Pᵢₙₗₑₜ at the valve inlet 208 exerts a force (Fᵢₙₗₑₜ) on the diaphragm 212 according to the following formula:

Fᵢₙₗₑₜ=Pᵢₙₗₑₜ*Aₛₑₐₜ

Where Fᵢₙₗₑₜ is the force on the diaphragm 212 from the valve inlet 208, Pᵢₙₗₑₜ is the pressure at the valve inlet 208, and Aₛₑₐₜ is an area defined by a diameter 224 of the valve seat 206.

In general, pressure is generated in the pilot pressure chamber 210 according to the formula:

Pₚᵢₗₒₜ=nRT/Vₚᵢₗₒₜ

Where Pₚᵢₗₒₜ is the pilot pressure in the pilot pressure chamber 210, Vₚᵢₗₒₜ is the volume of the pilot pressure chamber 210, n is the amount of gas in moles, R is the universal gas constant (8.314 J/mol·K), and T is the temperature.

In general, the pilot pressure Pₚᵢₗₒₜ in the pilot pressure chamber 210 exerts a force (Fₚᵢₗₒₜ) on the diaphragm 212 according to the following formula:

Fₚᵢₗₒₜ=Pₚᵢₗₒₜ*A_{diaphragm}

Where Fₚᵢₗₒₜ is the force on the diaphragm 212 from the pilot pressure chamber 210, Pₚᵢₗₒₜ is the pressure in the pilot pressure chamber 210, and A_{diaphragm} is defined by a diameter 226 of diaphragm 212. Thus, as illustrated, the seat area (dependent on the diameter 224 of valve seat 206) is not equal to the diaphragm area (dependent on the diameter 226 of the diaphragm 212).

According to embodiments, actuation of the valve is based on a force balance (i.e., opposing forces applied to the diaphragm 212). On one side, the pilot force (Fₚᵢₗₒ) is a product of the pilot pressure (Pₚᵢₗₒₜ), as created by the piezoelectric blower 218, and the diaphragm area (as defined by diameter 226). On the other side, the inlet force (F is a product of the inlet pressure (Pᵢₙₗₑₜ) and the valve seat area (as defined by diameter 224). If the inlet force (Fᵢₙₗₑₜ) created by the inlet pressure (Pᵢₙₗₑₜ) acting against the seat area of the diaphragm is less than the pilot force (Fₚᵢₗₒₜ) exerted by the pilot pressure acting against the diaphragm area, the pneumatic valve 200 will be closed with the diaphragm 212 pushed against the valve seat 206. If the inlet force (Fᵢₙₗₑₜ) created by the inlet pressure (Pᵢₙₗₑₜ) is greater than the pilot force (Fₚᵢₗₒₜ) created by the pilot pressure, the diaphragm 212 will be pushed away from the valve seat 206 and the pneumatic valve 200 will open and relieve pressure (e.g., via valve outlet 216). When these two forces are equal, the diaphragm 212 will come to rest at an equilibrium position.

Using the force balance, a change in pilot pressure (Pₚᵢₗₒₜ) can be used to control the inlet pressure (Pᵢₙₗₑₜ) level at which the pneumatic valve 200 will open and relieve/control pressure. If the pilot pressure (Pₚᵢₗₒₜ) is held constant, the pneumatic valve 200 will either close or open and relieve pressure based upon the dynamics of the inlet pressure (Pᵢₙₗₑₜ). As illustrated in FIG. 2, the force created by the pilot pressure (Pₚᵢₗₒₜ) is greater than the force (Fᵢₙₗₑₜ) created by the inlet pressure (Pᵢₙₗₑₜ) and the pneumatic valve 200 is in the closed position.

As described above, the seat area as defined by the diameter 224 of the valve seat 206 and the diaphragm area as defined by the diameter 226 of the diaphragm 212 are not the same. Because the diaphragm diameter 226 is greater than the seat diameter 224, the pilot pressure (Pₚᵢₗₒₜ) has a mechanical advantage over the inlet pressure (Pᵢₙₗₑₜ). That is, for a given pilot pressure (Pₚᵢₗₒₜ), the inlet pressure (Pᵢₙₗₑₜ) at which the pneumatic valve 200 will relieve pressure will be based upon the ratio of the two areas (Aₛₑₐₜ and A_{diaphragm}). For example, if the area of the diaphragm is 1.5 times greater than that of the valve seat, the relief pressure at the inlet will be 1.5 times the pilot pressure. By varying the pilot pressure, the pressure at which the valve inlet 208 will be controlled/relieved can be calculated by multiplying the pilot pressure by the ratio of the two areas (Aₛₑₐₜ and A_{diaphragm}).

According to embodiments, the pneumatic valve 200 may be implemented in different ways. For example, inlet pressure (Pᵢₙₗₑₜ) may be monitored and the piezoelectric blower 218 may be adjusted accordingly. Alternatively, the piezoelectric blower 218 may be characterized to determine a proper drive input to achieve a desired inlet pressure. Alternatively still, pilot pressure (Pₚᵢₗₒₜ) may be monitored, and the piezoelectric blower 218 may be controlled to achieve a desired pilot pressure (Pₚᵢₗₒₜ). The desired pilot pressure (Pₚᵢₗₒₜ) may be based upon the characteristic of the pneumatic valve 200 (e.g., the ratio of the seat and diaphragm areas). Moreover, various open and closed loop schemes for controlling the various pressures described above may be utilized.

As should be appreciated, pneumatic valve 200 is provided for illustrative purposes only. As such, placement and orientation of various components of pneumatic valve 200 are not exclusive and may be rearranged within the spirit of the present disclosure. For example, inlet ports and outlet ports may be placed in any suitable location within pneumatic valve 200. Moreover, piezoelectric blower 218 may be coupled to pneumatic valve 200 in any suitable location or orientation.

FIG. 3 is a diagram illustrating a piezoelectric blower coupled to a pneumatic valve in an open position.

As illustrated, pneumatic valve 300 comprises a valve housing 302 that surrounds an internal pneumatic valve chamber. The internal pneumatic valve chamber comprises an inlet chamber 304. The volume of the inlet chamber 304 is defined by a valve seat 306 and gas enters the inlet chamber 304 through valve inlet 308. According to embodiments, the internal pneumatic valve chamber further comprises a pilot pressure chamber 310. The pilot pressure chamber 310 is separated from the other chambers by a diaphragm 312 that is affixed within the pneumatic valve 300. According to embodiments, the internal pneumatic valve chamber further comprises an outlet chamber 314. When the pneumatic valve 300 is in an open position, gas entering the inlet chamber 304 is allowed to enter the outlet chamber 314. When the pneumatic valve 300 is in a closed position, gas entering the inlet chamber 304 is not allowed to enter the outlet chamber 314. Gas exits the outlet chamber 314 through a valve outlet 316.

According to further embodiments, pneumatic valve 300 includes a piezoelectric blower 318. According to embodiments, piezoelectric blower 318 is coupled to pneumatic valve 300 via any suitable means, e.g., via tubing, a connector, an interface, etc. According to some embodiments, piezoelectric blower 318 is closely coupled to pneumatic valve 300. That is, the piezoelectric blower 318 may be affixed substantially directly to pneumatic valve 300. According to embodiments, “affixed substantially directly” comprises any suitable gas-impermeable barrier or interface for closely coupling the piezoelectric blower to pneumatic valve 300. According to embodiments, gas enters piezoelectric blower 318 through a piezoelectric inlet port 320. Pressurized gas exits the piezoelectric blower 318 through a piezoelectric outlet port 322 that leads into the pilot pressure chamber 310.

As described above, an inlet pressure Pᵢₙₗₑₜ at the valve inlet 308 exerts a force (Fᵢₙₗₑₜ) on the diaphragm 312 according to the following formula:

Fᵢₙₗₑₜ=Pᵢₙₗₑₜ*Aₛₑₐₜ

Where Fᵢₙₗₑₜ is the force on the diaphragm 312 from the valve inlet 308, Pᵢₙₗₑₜ is the pressure at the valve inlet 308, and Aₛₑₐₜ is an area defined by a diameter of the valve seat 306.

In addition, the pilot pressure Pₚᵢₗₒₜ in the pilot pressure chamber 310 exerts a force (Fₚᵢₗₒₜ) on the diaphragm 312 according to the following formula:

Fₚᵢₗₒₜ=Pₚᵢₗₒₜ*A_{diaphragm}

Where Fₚᵢₗₒₜ is the force on the diaphragm 312 from the pilot pressure chamber 310, Pₚᵢₗₒₜ is the pressure in the pilot pressure chamber 310, and A_{diaphragm} is defined by the diameter of diaphragm 312. Thus, as illustrated, the seat area is not equal to diaphragm area.

According to embodiments, actuation of the valve is based on a force balance. If the force (Fᵢₙₗₑₜ) created by the inlet pressure (Pᵢₙₗₑₜ) acting against the seat area of the diaphragm is less than the force (Fₚᵢₗₒₜ) exerted by the pilot pressure acting against the diaphragm area, the pneumatic valve 300 will be closed with the diaphragm 312 pushed against the valve seat 306. If the force (Fᵢₙₗₑₜ) created by the inlet pressure (Pᵢₙₗₑₜ) is greater than the force (Fₚᵢₗₒₜ) created by the pilot pressure, the diaphragm 312 will be pushed away from the valve seat 306 and the pneumatic valve 300 will open and relieve pressure (e.g., via valve outlet 316). As illustrated in FIG. 3, the force (Fᵢₙₗₑₜ) created by the inlet pressure (Pᵢₙₗₑₜ) is greater than the force (Fₚᵢₗₒₜ) created by the pilot pressure and the pneumatic valve 300 is in the open position.

As should be appreciated, pneumatic valve 300 is provided for illustrative purposes only. As such, placement and orientation of various components of pneumatic valve 300 are not exclusive and may be rearranged within the spirit of the present disclosure. For example, inlet ports and outlet ports may be placed in any suitable location within pneumatic valve 300. Moreover, piezoelectric blower 318 may be coupled to pneumatic valve 300 in any suitable location or orientation.

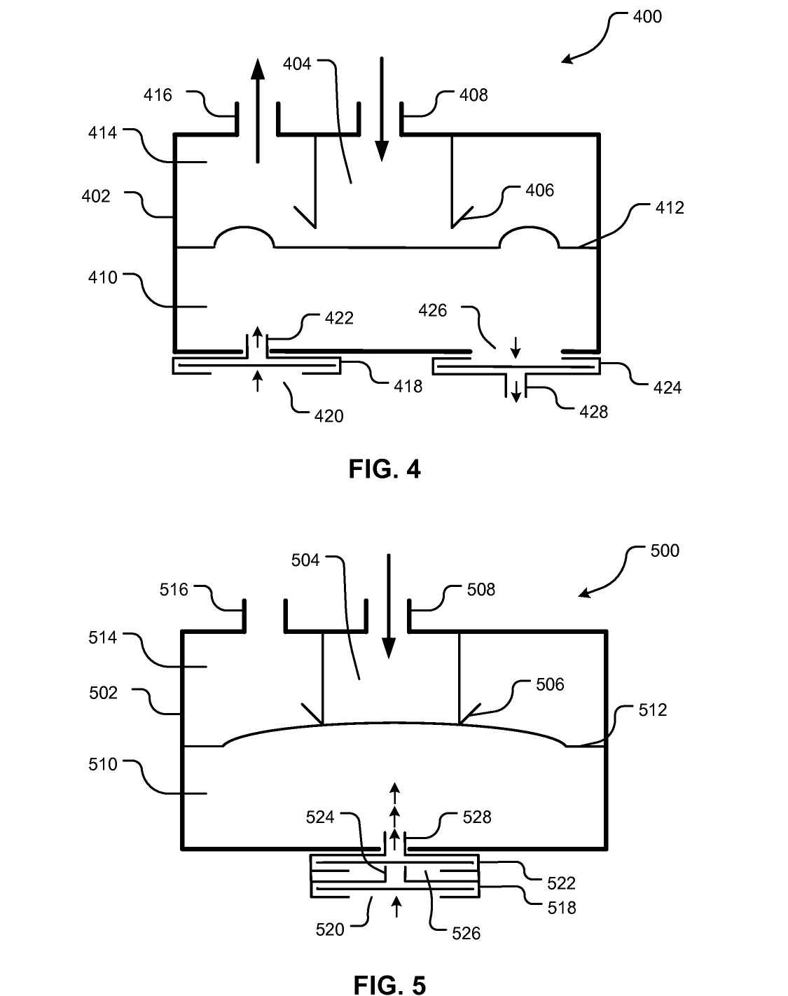

FIG. 4 is a diagram illustrating a plurality of piezoelectric blowers coupled in parallel to a pneumatic valve in an open position.

As illustrated, pneumatic valve 400 comprises a valve housing 402 that surrounds an internal pneumatic valve chamber. The internal pneumatic valve chamber comprises an inlet chamber 404. The volume of the inlet chamber 404 is defined by a valve seat 406 and gas enters the inlet chamber 404 through valve inlet 408. According to embodiments, the internal pneumatic valve chamber further comprises a pilot pressure chamber 410. The pilot pressure chamber 410 is separated from the other chambers by a diaphragm 412 that is affixed within the pneumatic valve 400. According to embodiments, the internal pneumatic valve chamber further comprises an outlet chamber 414. When the pneumatic valve 400 is in an open position, gas entering the inlet chamber 404 is allowed to enter the outlet chamber 414. When the pneumatic valve 400 is in a closed position, gas entering the inlet chamber 404 is not allowed to enter the outlet chamber 414. Gas exits the outlet chamber 414 through a valve outlet 416.

According to further embodiments, pneumatic valve 400 includes a first piezoelectric blower 418. According to embodiments, first piezoelectric blower 418 is coupled to pneumatic valve 400 via any suitable means, e.g., via tubing, a connector, an interface, etc. According to some embodiments, first piezoelectric blower 418 is closely coupled to pneumatic valve 400. That is, the first piezoelectric blower 418 may be affixed substantially directly to pneumatic valve 400. Similar to piezoelectric blowers 218 and 318, gas enters first piezoelectric blower 418 through a first piezoelectric inlet port 420. Pressurized gas exits the first piezoelectric blower 418 through a first piezoelectric outlet port 422 that leads into the pilot pressure chamber 410.

According to further embodiments, pneumatic valve 400 also includes a second piezoelectric blower 424. According to embodiments, second piezoelectric blower 424 is coupled to pneumatic valve 400. However, second piezoelectric blower 424 is oriented such that gas enters the second piezoelectric blower 424 from pilot pressure chamber 410 through a second piezoelectric inlet port 426. Pressurized gas exits the second piezoelectric blower 424 through a second piezoelectric outlet port 428 that releases gas from the pilot pressure chamber 410, e.g., to the atmosphere, to the expiratory limb of the patient tubing, to the expiratory module of the pneumatic system, or to'another chamber suitable for releasing gases from the pneumatic valve 400. As illustrated, the first piezoelectric blower 418 and the second piezoelectric blower 424 are arranged in parallel.

According to embodiments, a pilot pressure (Pₚᵢₗₒₜ) is generated in the pilot pressure chamber 406 that is a function of the net volume of gas entering the pilot pressure chamber 410 through first piezoelectric outlet port 418 and exiting the pilot pressure chamber 410 through second piezoelectric blower 424, the volume (Vₚᵢₗₒₜ) of pilot pressure chamber 410, and the temperature. According to embodiments, the pilot pressure (Pₚᵢₗₒₜ) in the pilot pressure chamber 410 may be increased by increasing the pressure generated by first piezoelectric blower 418 and/or by decreasing the pressure released by second piezoelectric blower 424. According to further embodiments, the pilot pressure (Pₚᵢₗₒₜ) in the pilot pressure chamber 410 may be decreased by decreasing the pressure generated by first piezoelectric blower 418 and/or by increasing the pressure released by second piezoelectric blower 424. Accordingly, the pilot pressure (Pₚᵢₗₒₜ) in the pilot pressure chamber 410 may be more quickly adjusted (increased or decreased) based on controlling both the first piezoelectric blower 418 and the second piezoelectric blower 424. As such, a response time for the pneumatic valve 400 may be correspondingly decreased.

According to the formulas identified above, the pilot pressure (Pₚᵢₗₒₜ) generated in the pilot pressure chamber 410 exerts a force (Fₚᵢₗₒₜ) on the diaphragm 412 that is a function of the area of the diaphragm 412. Moreover, for a given pilot pressure (Pₚᵢₗₒₜ), the inlet pressure (Pᵢₙₗₑₜ) at which the valve will relieve pressure will be based upon the ratio of the seat area (Aₛₑₐₜ) to the diaphragm area (A_{diaphragm}). By varying the pilot pressure, the pressure at which the valve inlet 408 will be controlled/relieved can be calculated by multiplying the pilot pressure by the ratio of the two areas (Aₛₑₐₜ and A_{diaphragm}). As illustrated in FIG. 4, the force determined by the pilot pressure (Pₚᵢₗₒₜ) is less than the force (Fᵢₙₗₑₜ) created by the inlet pressure (Pᵢₙₗₑₜ) and the pneumatic valve 400 is in the open position.

As should be appreciated, pneumatic valve 400 is provided for illustrative purposes only. As such, placement and orientation of various components of pneumatic valve 400 are not exclusive and may be rearranged within the spirit of the present disclosure. For example, inlet ports and outlet ports may be placed in any suitable location within pneumatic valve 400. Moreover, the first piezoelectric blower 418 and the second piezoelectric blower 424 may be coupled to pneumatic valve 400 in any suitable location or orientation.

FIG. 5 is a diagram illustrating a plurality of piezoelectric blowers coupled in series to a pneumatic valve in a closed position.

As illustrated, pneumatic valve 500 comprises a valve housing 502 that surrounds an internal pneumatic valve chamber. The internal pneumatic valve chamber comprises an inlet chamber 504. The volume of the inlet chamber 504 is defined by a valve seat 506 and gas enters the inlet chamber 504 through valve inlet 508. According to embodiments, the internal pneumatic valve chamber further comprises a pilot pressure chamber 510. The pilot pressure chamber 510 is separated from the other chambers by a diaphragm 512 that is affixed within the pneumatic valve 500. According to embodiments, the internal pneumatic valve chamber further comprises an outlet chamber 514. When the pneumatic valve 500 is in an open position, gas entering the inlet chamber 504 is allowed to enter the outlet chamber 514. When the pneumatic valve 500 is in a closed position, gas entering the inlet chamber 504 is not allowed to enter the outlet chamber 514. Gas exits the outlet chamber 514 through a valve outlet 516.

According to further embodiments, pneumatic valve 500 includes a first piezoelectric blower 518. Gas enters first piezoelectric blower 518 through a first piezoelectric inlet port 520. However, in this case, first piezoelectric blower 518 is coupled to a second piezoelectric blower 522. According to embodiments, the first piezoelectric blower 518 may be coupled to the second piezoelectric blower 522 via any suitable gas-impermeable barrier or other connecting means. According to embodiments, the second piezoelectric blower 522 is coupled to pneumatic valve 500.

As illustrated, the first piezoelectric blower 518 and the second piezoelectric blower 522 are arranged in series. In this case, pressurized gas exits the first piezoelectric blower 518 through a first piezoelectric outlet port 524 that leads into a second piezoelectric inlet port 526. Pressurized gas exits the second piezoelectric blower 522 through a second piezoelectric outlet port 528 that leads into the pilot pressure chamber 510. As illustrated, the combination of the first piezoelectric blower 518 and the second piezoelectric blower 522 generates a higher gas pressure than a single piezoelectric blower (e.g., piezoelectric blowers 218 and 318) (illustrated by triple arrows through second piezoelectric outlet port 528).

According to embodiments, a pilot pressure (Pₚᵢₗₒₜ) is generated in the pilot pressure chamber 510 that is a function of the volume of gas entering the pilot pressure chamber 510 through second piezoelectric outlet port 528, the volume (Vₚᵢₗₒₜ) of pilot pressure chamber 510, and the temperature. According to embodiments, the pilot pressure (Pₚᵢₗₒₜ) in the pilot pressure chamber 510 may be increased by arranging a plurality of piezoelectric blowers in series. That is, the pilot pressure (Pₚᵢₗₒₜ) in the pilot pressure chamber 510 is a function of the pressure generated by both the first piezoelectric blower 518 and the second piezoelectric blower 522. As such, a pressure attainable within the pilot pressure chamber 510 may be increased by arranging a plurality of piezoelectric blowers in series.

According to the formulas identified above, the pilot pressure (Pₚᵢₗₒₜ) generated in the pilot pressure chamber 510 exerts a force (Fₚᵢₗₒₜ) on the diaphragm 512 that is a function of the area of the diaphragm 512. As described above, for a given pilot pressure (Pₚᵢₗₒₜ), the inlet pressure (Pᵢₙₗₑₜ) at which the valve will relieve pressure will be based upon the ratio of the seat area (Aₛₑₐₜ) to the diaphragm area (A_{diaphragm}). By varying the pilot pressure, the pressure at which the valve inlet 508 will be controlled/relieved can be calculated by multiplying the pilot pressure by the ratio of the two areas (Aₛₑₐₜ and A_{diaphragm}). As illustrated in FIG. 5, the force determined by the pilot pressure (Pₚᵢₗₒₜ) is greater than the force (Fᵢₙₗₑₜ) created by the inlet pressure (Pᵢₙₗₑₜ) and the pneumatic valve 500 is in the closed position.

As should be appreciated, pneumatic valve 500 is provided for illustrative purposes only. As such, placement and orientation of various components of pneumatic valve 500 are not exclusive and may be rearranged within the spirit of the present disclosure. For example, inlet ports and outlet ports may be placed in any suitable location within pneumatic valve 500. Moreover, first piezoelectric blower 518 and second piezoelectric blower 522 may be coupled to pneumatic valve 500 in any suitable location. Moreover, a plurality of piezoelectric blowers may be coupled to a pneumatic valve in series and in parallel to increase both the pilot pressure attainable and the response time for piloting the pneumatic valve. As used herein, “piloting” a pneumatic valve comprises increasing and decreasing the pilot pressure in a pilot pressure chamber in order to open and close the pneumatic valve. According to embodiments, any suitable number of piezoelectric blowers may be coupled in series and/or in parallel to a pneumatic valve in order to precisely and quickly regulate the pilot pressure.

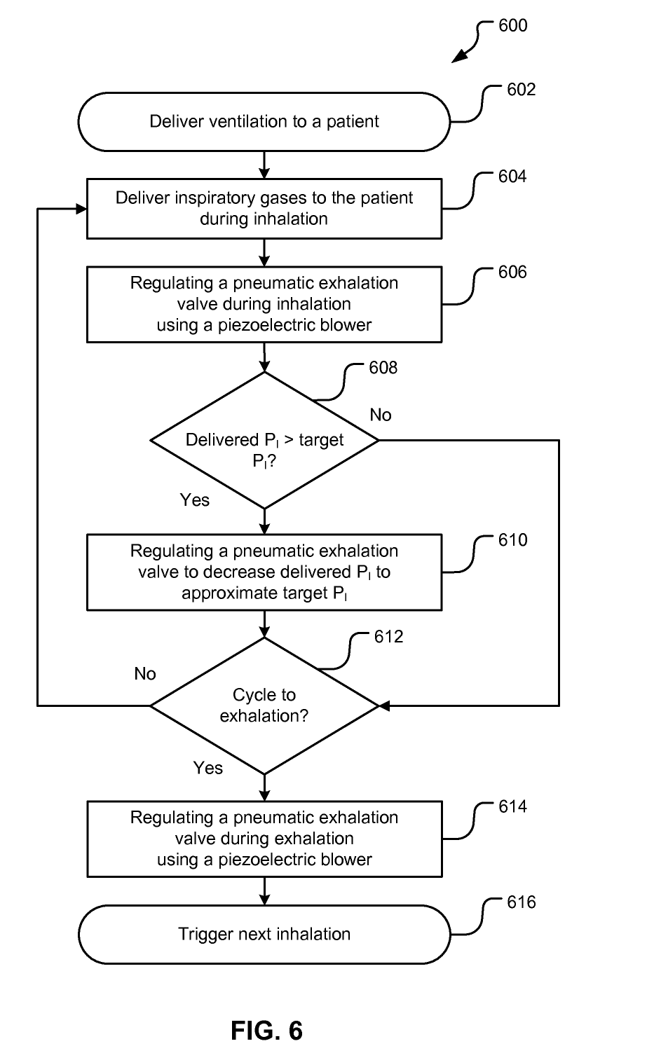

FIG. 6 is a flow chart illustrating an embodiment of a method for delivering ventilation to a patient using an exhalation valve piloted with a piezoelectric blower.

Method 600 begins with deliver ventilation operation 602. At deliver ventilation operation 602, a ventilator delivers breathing gases to a patient. The ventilator may deliver breathing gases to the patient based on a plurality of settings and parameters. According to embodiments, the ventilator may be configured to deliver gases to the patient during an inspiratory phase of ventilation and may be configured to release exhaled gases from the patient during an expiratory phase of ventilation. According to embodiments, the ventilator may be further configured to trigger inspiration (e.g., based on a set inspiratory time or based on a patient-initiated trigger) and to cycle exhalation (e.g., based on a set expiratory time, based on satisfaction of one or more cycling conditions, etc.).

At deliver operation 604, the ventilator provides inspiratory gases to a patient. According to embodiments, the ventilator may be configured with a target inspiratory pressure (P) for delivery to a patient, e.g., via input from a clinician, as determined by an appropriate protocol, etc. Alternatively, the ventilator may be configured with a tidal volume for delivery to a patient, e.g., via input from a clinician, as determined by an appropriate protocol, etc.

At regulate operation 606, an exhalation valve is regulated by the ventilator during inhalation. According to embodiments, the exhalation valve is a pneumatic valve that is coupled to a piezoelectric blower. According to embodiments, the pneumatic exhalation valve is substantially closed during inhalation so that inspiratory gases may be delivered to the patient. According to embodiments, actuation of the valve is based on a force balance. On one side, the pilot force is a product of the pilot pressure, as created by a piezoelectric blower, and the diaphragm area. On the other side, the inlet force is a product of the inlet pressure and the valve seat area. If the inlet force created by the inlet pressure acting against the seat area of the diaphragm is less than the pilot force exerted by the pilot pressure acting against the diaphragm area, the pneumatic exhalation valve will be closed with the diaphragm pushed against the valve seat. Accordingly, inspiratory gases are delivered to the patient and are prevented from being released through the pneumatic exhalation valve.

At decision operation 608, the ventilator determines whether a delivered inspiratory pressure is greater than a target inspiratory pressure during inhalation. The ventilator may determine whether the delivered inspiratory pressure is greater than the target inspiratory pressure via any suitable means. For example, the ventilator may detect a pressure in the patient tubing, a pressure at the wye interface, a pressure at an invasive or non-invasive interface of the patient, a pressure at the pneumatic exhalation valve, etc. When the delivered inspiratory pressure is not greater than the target inspiratory pressure (or when the ventilator is configured to deliver a tidal volume rather than a target inspiratory pressure), the method proceeds to decision operation 612. Alternatively, when the delivered inspiratory pressure is greater than the target inspiratory pressure, the method proceeds to regulate operation 610.

At regulate operation 610, the ventilator regulates the pneumatic exhalation valve in order to release excess pressure such that the delivered inspiratory pressure is not greater than the target inspiratory pressure. According to embodiments, excess pressure is released when the pneumatic exhalation valve is at least partially open. As described above, if the force (Fᵢₙₗₑₜ) created by the inlet pressure (Pᵢₙₗₑₜ) acting against the seat area of the diaphragm is less than the force (Fₚᵢₗₒₜ) exerted by the pilot pressure, the valve will be closed with the diaphragm pushed against the valve seat. If the force (Fᵢₙₗₑₜ) created by the inlet pressure (Pᵢₙₗₑₜ) is greater than the force (Fₚᵢₗₒₜ) created by the pilot pressure, the diaphragm will be pushed away from the valve seat and the valve will open and relieve pressure. Using the force balance, a change in pilot pressure (Pₚᵢₗₒₜ) can be used to control the inlet pressure (Pᵢₙₗₑₜ) level at which the valve will open and relieve/control pressure. If the pilot pressure (Pₚᵢₗₒₜ) is held constant, the valve will either close or open and relieve pressure based upon the dynamics of the inlet pressure (Pᵢₙₗₑₜ). Accordingly, if the inlet force (Fᵢₙₗₑₜ) is greater than the pilot force (Fₚᵢₗₒₜ), the diaphragm will be pushed away from the valve seat and the valve will open to relieve pressure such that delivered inspiratory pressure is not greater than the target inspiratory pressure.

At decision operation 612, the ventilator determines whether to cycle to an exhalation phase. The ventilator may determine whether to cycle to the exhalation phase via any suitable means. For example, the ventilator may cycle to the exhalation phase based on reaching a set inspiratory time, based on detecting that one or more cycling conditions have been satisfied, or otherwise. When the ventilator determines to cycle to the exhalation phase, the method proceeds to regulate operation 614. Alternatively, when the ventilator determines not to cycle to the exhalation phase, the method returns to deliver operation 604.

At regulate operation 614, the ventilator regulates the pneumatic exhalation valve in order to release exhaled gases. According to embodiments, exhaled gases are released when the pneumatic exhalation valve is substantially open. As described above, if the inlet force (Fᵢₙₗₑₜ) is greater than the pilot force (Fₚᵢₗₒₜ), the diaphragm will be pushed away from the valve seat and the valve will open to relieve pressure in order to release exhaled gases.

At trigger operation 616, the ventilator triggers a next inspiration. According to embodiments, the ventilator may trigger the next inspiration via any suitable means, e.g., detecting the end of an expiratory time, detecting spontaneous inspiratory effort by the patient, or otherwise.

As should be appreciated, the particular steps and methods described above with reference to FIG. 6 are not exclusive and, as will be understood by those skilled in the art, the particular ordering of steps as described herein is not intended to limit the method, e.g., steps may be performed in differing order, additional steps may be performed, and disclosed steps may be excluded without departing from the spirit of the present methods. Indeed, there are many different embodiments that could be used to deliver a breath and control the operation of the valve, using both open loop and close loop controls means.

Those skilled in the art will recognize that the methods and systems of the present disclosure may be implemented in many manners and as such are not to be limited by the foregoing exemplary embodiments and examples. In other words, functional elements being performed by a single or multiple components, in various combinations of hardware and software or firmware, and individual functions, can be distributed among software applications at either the client or server level or both. In this regard, any number of the features of the different embodiments described herein may be combined into single or multiple embodiments, and alternate embodiments having fewer than or more than all of the features herein described are possible. Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software or firmware components described herein as would be understood by those skilled in the art now and hereafter.

Numerous other changes may be made which will readily suggest themselves to those skilled in the art and which are encompassed in the spirit of the disclosure and as defined in the appended claims. While various embodiments have been described for purposes of this disclosure, various changes and modifications may be made which are well within the scope of the present invention. Numerous other changes may be made which will readily suggest themselves to those skilled in the art and which are encompassed in the spirit of the disclosure and as defined in the appended claims.

What is claimed is:

1. A pneumatic valve comprising:
a valve housing surrounding an internal pneumatic valve chamber, the internal pneumatic valve chamber divided by a diaphragm into a plurality of chambers comprising:
an inlet chamber having a valve inlet for receiving gases, the inlet chamber having an inlet pressure exerting an inlet force on the diaphragm; and
a pilot pressure chamber coupled to a piezoelectric outlet port for receiving gases, the pilot pressure chamber having a pilot pressure exerting a pilot force on the diaphragm;
a piezoelectric blower coupled to the pneumatic valve, the piezoelectric blower having a piezoelectric inlet port for receiving gases and the piezoelectric outlet port for delivering pressurized gases to the pilot pressure chamber;
a valve seat disposed within the inlet chamber; and
the diaphragm flexibly displaced based on the pilot force and the inlet force.

2. The pneumatic valve of claim 1, wherein the piezoelectric blower further comprises a piezoelectric crystal, wherein the piezoelectric crystal vibrates in response to an electric current, and wherein a pressure of gases delivered to the pilot pressure chamber is based on a speed of vibration of the piezoelectric crystal.

3. The pneumatic valve of claim 2, wherein a higher pressure of gases is delivered to the pilot pressure chamber when the speed of vibration of the piezoelectric crystal is higher, and wherein a lower pressure of gases is delivered to the pilot pressure chamber when the speed of vibration of the piezoelectric crystal is lower.

4. The pneumatic valve of claim 1, wherein the piezoelectric blower is closely coupled to the pneumatic valve.

5. The pneumatic valve of claim 1, wherein the diaphragm is flexibly displaced away from the valve seat to open the pneumatic valve when the pilot force is less than the inlet force.

6. The pneumatic valve of claim 1, wherein the diaphragm is flexibly displaced toward the valve seat to close the pneumatic valve when the pilot force is greater than the inlet force.

7. The pneumatic valve of claim 1, the pneumatic valve further comprising one or more additional piezoelectric blowers.

8. The pneumatic valve of claim 7, wherein the one or more additional piezoelectric blowers are coupled to the pneumatic valve in a parallel arrangement to the piezoelectric blower.

9. The pneumatic valve of claim 7, wherein the one or more additional piezoelectric blowers are coupled to the pneumatic valve in a series arrangement to the piezoelectric blower.

10. A method for delivering ventilation to a patient, comprising:
delivering inspiratory gases to a patient during an inspiratory phase;
regulating a pneumatic exhalation valve during the inspiratory phase, comprising:
receiving gases into an inlet chamber of the pneumatic exhalation valve, wherein an inlet pressure exerts an inlet force on the diaphragm of the pneumatic exhalation valve based on an area of a valve seat;
controlling a piezoelectric blower to deliver pressurized gases to a pilot pressure chamber, wherein a pilot pressure exerts a pilot force on the diaphragm of the pneumatic exhalation valve based on an area of the diaphragm; and
substantially closing the pneumatic exhalation valve when the pilot force is greater than the inlet force.

11. The method of claim 10, wherein the piezoelectric blower is closely coupled to the pneumatic exhalation valve.

12. The method of claim 11, wherein the pneumatic exhalation valve is a proximal pneumatic exhalation valve located substantially near the patient.

13. The method of claim 12, wherein the pneumatic exhalation valve is a proximal pneumatic exhalation valve located in a non-invasive patient interface.

14. The method of claim 10, wherein controlling the piezoelectric blower to deliver pressurized gases to the pilot pressure chamber further comprises:
causing the piezoelectric crystal to vibrate in response to an electric current, wherein a gas pressure of the pilot pressure chamber is based on a speed of vibration of the piezoelectric crystal, wherein a higher gas pressure is delivered to the pilot pressure chamber when the speed of vibration of the piezoelectric crystal is higher, and wherein a lower gas pressure is delivered to the pilot pressure chamber when the speed of vibration of the piezoelectric crystal is lower.

15. The method of claim 10, further comprising:
cycling to an exhalation phase; and
regulating the exhalation valve during the exhalation phase, comprising:
controlling a piezoelectric blower to deliver pressurized gases to a pilot pressure chamber, wherein a pilot pressure exerts a pilot force on the diaphragm of the pneumatic exhalation valve based on an area of the diaphragm; and
substantially opening the pneumatic exhalation valve when the pilot force is less than the inlet force.

16. A pneumatic valve means comprising:
a valve housing means surrounding an internal pneumatic valve chamber, the internal pneumatic valve chamber divided by a diaphragm means into a plurality of chambers comprising:
an inlet chamber having a valve inlet for receiving gases, the inlet chamber having an inlet pressure exerting an inlet force on the diaphragm means; and
a pilot pressure chamber coupled to a piezoelectric outlet port for receiving gases, the pilot pressure chamber having a pilot pressure exerting a pilot force on the diaphragm means;
a piezoelectric blower means coupled to the pneumatic valve means, the piezoelectric blower means having a piezoelectric inlet port for receiving gases and the piezoelectric outlet port for delivering pressurized gases to the pilot pressure chamber;
a valve seat means disposed within the inlet pressure chamber; and
the diaphragm means flexibly displaced based on the pilot force and the inlet force.

17. The pneumatic valve means of claim 16, wherein the piezoelectric blower means further comprises a piezoelectric crystal means, wherein the piezoelectric crystal means vibrates in response to an electric current, and wherein a pressure of gases delivered to the pilot pressure chamber is based on a speed of vibration of the piezoelectric crystal means.

18. The pneumatic valve means of claim 17, wherein a higher pressure of gases is delivered to the pilot pressure chamber when the speed of vibration of the piezoelectric crystal means is higher, and wherein a lower pressure of gases is delivered to the pilot pressure chamber when the speed of vibration of the piezoelectric crystal means is lower.

19. The pneumatic valve means of claim 16, wherein the diaphragm means is flexibly displaced away from the valve seat means to open the pneumatic valve means when the pilot force is less than the inlet force.

20. The pneumatic valve means of claim 16, wherein the diaphragm means is flexibly displaced toward the valve seat means to close the pneumatic valve means when the pilot force is greater than the inlet force.
